(19) United States
(12) Patent Application Publication
(10) Pub. No.: US 2012/0109070 A1
(43) Pub. Date: May 3, 2012

(54) CATHETER SECUREMENT DEVICE TO SECURE SILICONE WINGED PICCS

(75) Inventors: Tamer Elsamahy, Brighton, MI (US); Brian Archipley, Okemos, MI (US)

(73) Assignee: CENTURION MEDICAL PRODUCTS CORPORATION, Williamston, MI (US)

(21) Appl. No.: 12/914,242

(22) Filed: Oct. 28, 2010

(51) Int. Cl. A61M 25/02

(52) U.S. Cl. 604/179; 604/180

(57) ABSTRACT

A catheter securement device in accordance with the present invention includes a sheet-like anchoring member having a top surface, an opposite bottom surface, a base portion, and a pair of strap portions extending from the base portion. The anchoring member includes an adhesive on the bottom surface in the base portion and is generally adhesive free on the top surface in the base portion. The anchoring member also includes an adhesive on the top surface in the strap portions and is generally adhesive free on the bottom surface in the strap portions. The base portion is mountable on a patient's skin intermediate a catheter hub having wings, and each strap portion is foldable over the base portion and one catheter hub wing to secure the catheter hub.

### TECHNICAL FIELD

This invention relates to catheter securement devices, and more particularly to securement devices for securing catheters to a patient's skin.

### BACKGROUND OF THE INVENTION

It is known in the art relating to catheters that after insertion of a catheter through a patient's skin into a vein, the catheter must be secured to prevent the catheter from slipping out of the patient's vein. Commonly, surgical tape is used to hold the catheter hub or tubing connected to the catheter to the patient's skin. Oftentimes, this method is ineffective to permanently, securely anchor the catheter and to prevent catheter movement.

Further, inadvertent movement of a catheter while the catheter is inserted in a vein is a leading cause of premature catheter failure. When a catheter moves in a vein, it scrapes and pokes the inner wall of the vein, thereby irritating the vein. Repeated movement of the catheter thereby causes sufficient irritation of the vein to require the catheter to be removed and a new catheter inserted in a different location along the same vein or in an entirely new vein. This is costly as it results in a waste of resources. Also, repeated movement of an inserted catheter can cause migration of the catheter in the vein or worse, may lead to the catheter being removed from the vein. Therefore, a need exists for effective anchoring/securement devices for catheters.

Moreover, it is also know to use a catheter dressing with pressure sensitive adhesives to fully secure and protect a catheter such as a Peripherally Inserted Central Catheter (“PICC”). PICCs are typically made of polyurethane or silicone based materials. Due to the low surface energy of silicones, adhesives from dressings do not fully anchor or grip to silicone substrates as well as polyurethane materials. Thus, PICC movement will occur when a PICC covered with a dressing is tugged or pulled.

### SUMMARY OF THE INVENTION

The present invention provides a catheter securement device that securely anchors a catheter to a patient's skin while at the same time is easily removed from the catheter without damaging the catheter or disturbing the catheter insertion site. The present catheter securement device is designed to be used to anchor catheters having wings, such as suture wings extending from a hub of the catheter. For example, one intended use of the present catheter securement device is to secure PICCs (Peripherally Inserted Central Catheters), but the catheter securement device may be used to secure any winged-type catheter. The present catheter securement device prevents stresses applied to the catheter hub or luer connectors attached to the catheter from transferring to the catheter insertion site and causing catheter migration or removal as well as insertion site irritation.

More particularly, a catheter securement device in accordance with the present invention includes a sheet-like anchoring member having a top surface, an opposite bottom surface, a base portion, and a pair of strap portions extending from the base portion. The anchoring member includes an adhesive on the bottom surface in the base portion and is generally adhesive free on the top surface in the base portion. The anchoring member also includes an adhesive on the top surface in the strap portions and is generally adhesive free on the bottom surface in the strap portions. The base portion is mountable on a patient's skin intermediate a catheter hub having wings, and each strap portion is foldable over the base portion and one catheter hub wing to secure the catheter hub.

Each strap portion may include a leg adjacent the base portion and a distal, outwardly extending foot. The anchoring member may include a perforated line generally bisecting the base portion for tear removal of the anchoring member. The anchoring member may have a generally wishbone-like shape. The anchoring member may be generally symmetrical about one axis.

The catheter securement device may include a first release liner disposed adjacent to the bottom surface of the anchoring member. The first release liner may extend beyond the anchoring member. The catheter securement device also may include second and third release liners disposed adjacent to the top surface of the anchoring member. The second and third release liners each may cover the adhesive on one of the strap portions. The second and third release liners each may generally correspond in shape to one of the strap portions. The second and third release liners each may include a free tab portion that is folded over a portion that covers the strap portions.

The anchoring member may include a fold line generally dividing the base portion from the strap portions. Each strap portion may be foldable over the base portion along the fold line. The anchoring member may include a foam layer and an adjacent fabric layer.

These and other features and advantages of the invention will be more fully understood from the following detailed description of the invention taken together with the accompanying drawings.

### BRIEF DESCRIPTION OF THE DRAWINGS

In the drawings:

FIG. 1 is a perspective view of a catheter securement device in accordance with the present invention;

FIG. 2 is a cross-sectional view of the catheter securement device taken along the line 2-2 in FIG. 1;

FIG. 3 is an environmental view of the catheter securement device of FIG. 1 adhered to a patient's skin, and illustrating a method of anchoring a catheter hub in which the catheter securement device has been positioned underneath the catheter hub;

FIG. 4 is an environmental view illustrating the method of anchoring the catheter hub in which one strap portion has secured one catheter wing of the catheter hub; and

FIG. 5 is an environmental view illustrating the method of anchoring the catheter hub in which the other strap portion has secured the other catheter wing of the catheter hub.

### DETAILED DESCRIPTION OF THE INVENTION

Referring now to the drawings in detail, numeral 10 generally indicates a catheter securement device in accordance with the present invention. A bottom side of the securement device 10 can be adhered to a patient's skin. The securement device 10 then anchors a catheter by straping catheter hub wings of a catheter inserted into a patient, thereby harnessing the catheter hub. Once the catheter is secured by the securement device 10, stresses and forces applied in any direction on luers and fittings connected to the catheter will not cause the catheter to move and thereby irritate the insertion site. The catheter wings are also easily removable from the securement device 10, allowing for discarding of the securement device without disturbing the catheter inserted in the insertion site.

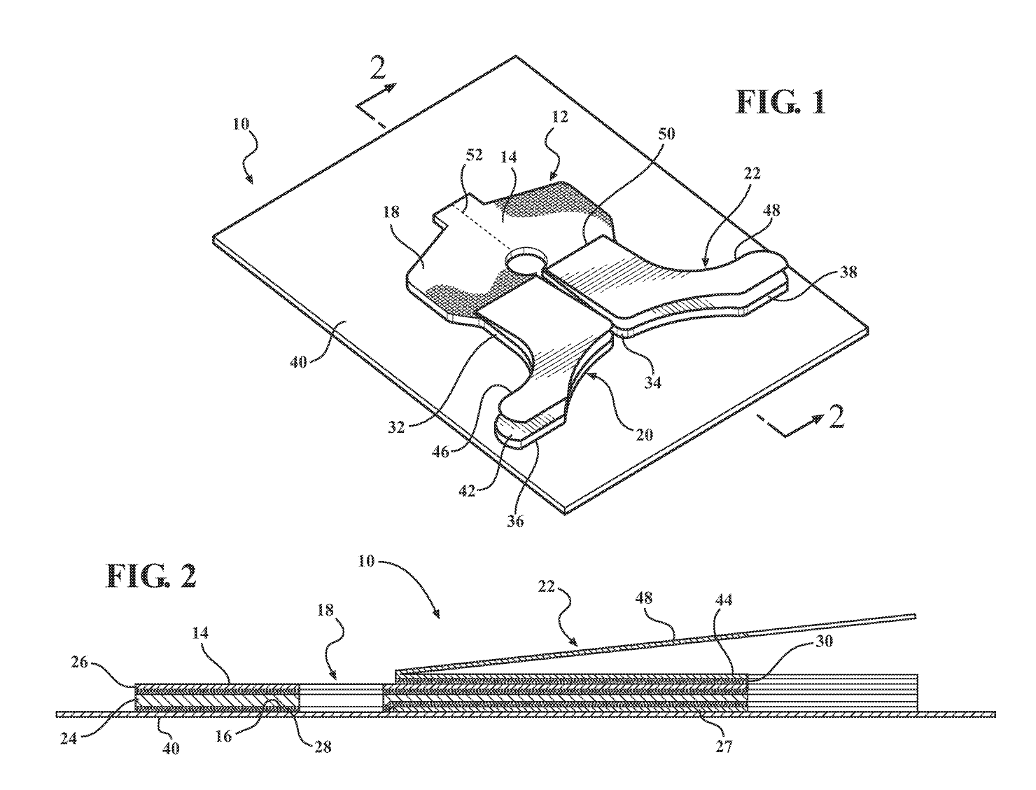

Turning first to FIGS. 1 and 2, a catheter securement device 10 in accordance with the present invention includes a sheet-like, single piece anchoring member 12 having a top surface 14, an opposite bottom surface 16, a base portion 18, and a pair of strap portions 20, 22 extending from the base portion 18. The anchoring member 12 may be a single layer of material, or alternatively may include a lower foam layer 24 and an adjacent upper fabric layer 26, or a multi-laminate of film, foam and fabric may be constructed. The anchoring member 12 may have a generally wishbone-like shape and may be generally symmetrical about one of its axes.

The anchoring member 12 includes an adhesive layer 28 on the bottom surface 16 in the base portion 18, and is generally adhesive free on the top surface 14 in the base portion 18. The anchoring member 12 also includes an adhesive layer 30 on the top surface 14 in the strap portions 20, 22, and is generally adhesive free on the bottom surface 16 in the strap portions 20, 22. An additional fabric layer 27 having an adhesive free outer surface may be adhered to the foam layer 24 in the strap portions 20, 22 to provide for the adhesive free bottom surface 16 of the strap portions. The fabric layer 27 may be a low surface energy fabric material that importantly prevents a dressing placed over the device 10 (in use on a patient's skin) from sticking to the device upon removal. Alternatively, the bottom side of the foam layer 26 may be free of adhesive. Each strap portion 20, 22 may include a leg 32, 34, respectively, that is adjacent the base portion 18. Each strap portion 20, 22 also may include a distal, outwardly extending foot 36, 38, respectively.

A first release liner 40 may be disposed adjacent to the bottom surface 16 of the anchoring member 12 to protect the adhesive layer 28 from inadvertently sticking to an object prior to use. The first release liner 40 has a tackless side such as a siliconized surface, a wax surface, or similar that contacts the adhesive layer 28 but is easily peeled away from the adhesive layer 28 without removing the adhesive from the anchoring member 12. The first release liner 40 may extend beyond the outer edge of the anchoring member 12. A second release liner 42 and a third release liner 44 may be disposed adjacent to the top surface 14 of the anchoring member 12. The second release liner 42 has a tackless side that contacts and covers the adhesive layer 30 on strap portion 20, and the third release liner 44 has a tackless side that contacts and covers the adhesive layer 30 on strap portion 22. The second and third release liners 42, 44 each may generally correspond in shape to their respective strap portions 20, 22. Also, the second and third release liners 42, 44 each may include a free tab portion 46, 48, respectively, that is folded over the portion of the release liner that contacts and covers the strap portions 20, 22. The tab portions 46, 48 may have the same shape as the portions of the second and third release liners 42, 44 that cover the adhesive on the strap portions 20, 22.

The anchoring member 12 may include a fold line 50 that generally dividing the base portion 18 from the strap portions 20, 22. Each strap portion 20, 22 is foldable over the base portion 18 along the fold line, as described in more detail below. The anchoring member 12 also may include a perforated tear line 52 that generally bisects the base portion for tear removal and disposal of the anchoring member after it has been used. The tear line 52 may generally lie along the axis of symmetry of the anchoring member 12.

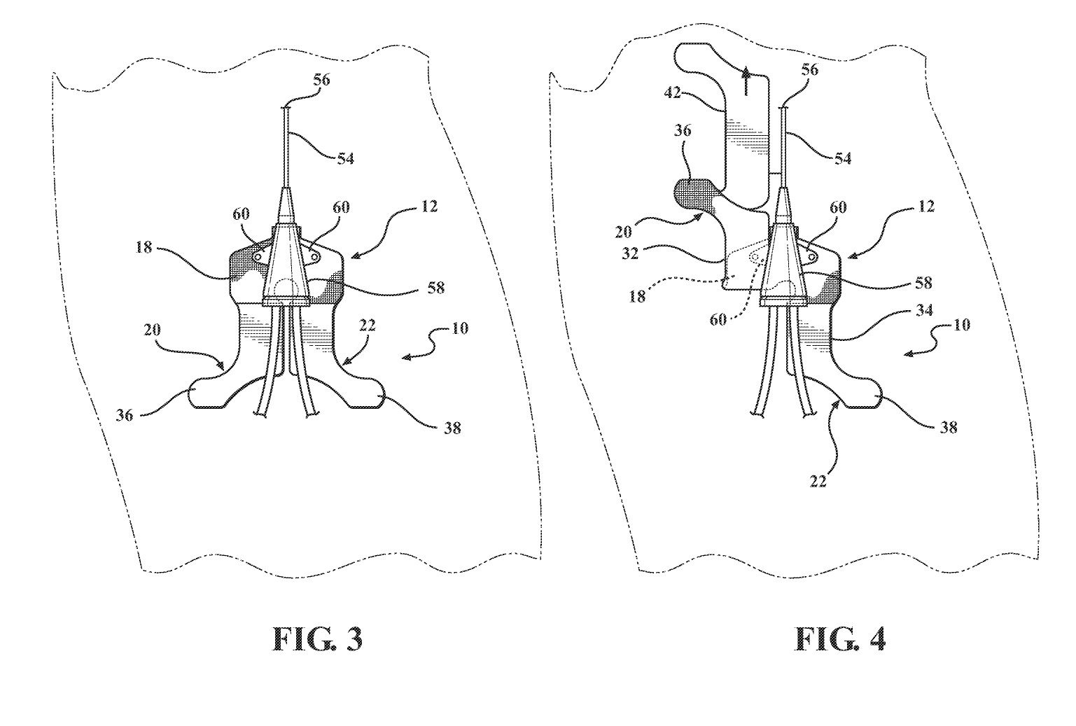

Turning to FIGS. 1 through 5, for purposes of example the securement device 10 is shown securing a PICC 54 (Peripherally Inserted Central Catheter). First, a health care provider, herein a nurse for illustrative purposes, inserts the catheter 54 through a patient's skin at an insertion site 56. After insertion of the catheter 54, the nurse removes the first release liner 40 that covers the bottom surface 16 of the anchoring member 12. This exposes the adhesive layer 28 on the base portion 18, which the nurse mounts on the patient's skin such that the base portion 18 is positioned below/underneath the catheter hub 58 and catheter hub wings 60 that extend from either side of the catheter hub as shown in FIG. 3. The anchoring member 12 is also positioned so that the strap portions 20, 22 face away from the insertion site 56.

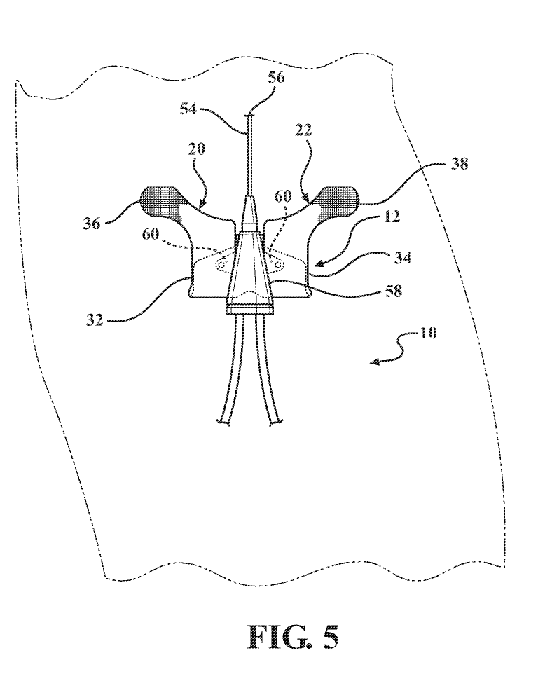

Next, the nurse grips the tab portion 46 of the second release liner 42 and begins to pull the second release liner 42 away from the strap portion 20. As the nurse pulls the second release liner 42 away, he/she may pull the second release liner 42 in the direction of the insertion site 56 towards the catheter hub wing 60. This movement causes the strap portion 20 to pivot about the fold line 50 and draws the strap portion 20 over the catheter hub wing 60 without the nurse having to touch the adhesive 30 on the strap portion. The strap portion 20 is then secured over the catheter hub wing 60 by pressing on the adhesive free bottom surface 16 of the strap portion 20 which now faces upwards/away from the base portion 18 and the patient's skin as shown in FIG. 4. The leg 32 of the strap portion 20 generally straps the hub wing 60 while the foot 36 may at least partially adhere directly to the patient's skin. Similarly, the nurse grips the tab portion 48 of the third release liner 42 and begins to pull the third release liner 44 away from the strap portion 22. The nurse pulls the third release liner 44 in the direction of the insertion site 56 towards the other catheter hub wing 60, pivoting the strap portion 22 about the fold line 50 and drawing the strap portion 22 over the other catheter hub wing 60. The strap portion 22 is then secured over the other catheter hub wing 60 as shown in FIG. 5. The leg 34 of the strap portion 22 generally straps the hub wing 60 while the foot 38 may at least partially adhere directly to the patient's skin.

In this arrangement, the catheter securement device 10 holds the catheter hub 58 in place and prevents movement of the catheter 54 when pulling or tugging forces are exerted on the lumen/cannula/tubing extending from the hub 58. For example, the catheter securement device 10 may restrain movement of the catheter up to 4 times or more than if no securement device is used (e.g., just a dressing applied over the hub). The device 10 may also include a generally arcuate cut-out generally centrally located between the base portions 18 and the strap portions 20, 22. When the strap portions 20, 22 are folded over the catheter wings 60 as described above, the cut-out portion creates an opening or recess that allows an antimicrobial disk or patch to mounted comfortably around the catheter insertion site with no interference by the device 10.

After the catheter securement device 10 has secured the catheter hub 58, a dressing may be applied over the insertion site 56 and secured catheter hub 58 to further protect the insertion site. The securement device 10 also provides a buffer between the non-breathable material of the catheter hub 58 and the patient's skin. Since the material(s) of the securement device 10 are breathable, more moisture can be released from the area around the catheter hub 58 than if the catheter hub were laying over and contacting the skin. The securement device 10 also increases patient comfort, acting as a cushion that shields the skin from the hard material and/or coarse edges of the catheter hub 58.

To remove the securement device 10 from the catheter hub 58, the nurse peels each of the strap portions 20, 22 away from the skin. Then, while the nurse grasps the strap portions 20, 22 in separate hands, he/she pulls the strap portions away from each other and with a “wishbone” action, the securement device 10 separates along the tear line 52 and can be easily moved out from under the catheter hub 58 without disturbing the catheter hub or inserted catheter 54.

Although the invention has been described by reference to a specific embodiment, it should be understood that numerous changes may be made within the spirit and scope of the inventive concepts described. Accordingly, it is intended that the invention not be limited to the described embodiment, but that it have the full scope defined by the language of the following claims.

What is claimed is:

1. A catheter securement device comprising:
a sheet-like anchoring member including a top surface, an opposite bottom surface, a base portion, and a pair of strap portions extending from said base portion;
said anchoring member including an adhesive on said bottom surface in said base portion and being generally adhesive free on said top surface in said base portion;
said anchoring member including an adhesive on said top surface in said strap portions and being generally adhesive free on said bottom surface in said strap portions;
wherein said base portion is mountable on a patient's skin intermediate a catheter hub having wings, and each said strap portion is foldable over said base portion and one said catheter hub wing to secure said catheter hub.

2. The catheter securement device of claim 1, wherein each said strap portion includes a leg adjacent said base portion and a distal, outwardly extending foot.

3. The catheter securement device of claim 1, wherein said anchoring member includes a perforated line generally bisecting said base portion for tear removal of the anchoring member.

4. The catheter securement device of claim 1, wherein said anchoring member has a generally wishbone-like shape.

5. The catheter securement device of claim 1, wherein said anchoring member is generally symmetrical about one axis.

6. The catheter securement device of claim 1, including a first release liner disposed adjacent to said bottom surface of said anchoring member.

7. The catheter securement device of claim 6, wherein said first release liner extends beyond said anchoring member.

8. The catheter securement device of claim 1, including second and third release liners disposed adjacent to said top surface of said anchoring member, said second and third release liners each covering the adhesive on one said strap portion.

9. The catheter securement device of claim 8, wherein said second and third release liners each generally corresponds in shape to one of said strap portions.

10. The catheter securement device of claim 9, wherein said second and third release liners each include a free tab portion that is folded over a portion that covers the strap portions.

11. The catheter securement device of claim 1, wherein said anchoring member includes a fold line generally dividing the base portion from the strap portions, wherein each said strap portion is foldable over said base portion along said fold line.

12. The catheter securement device of claim 1, wherein said anchoring member includes a foam layer and an adjacent fabric layer.
